(19) United States
(12) Patent Application Publication
(10) Pub. No.: US 2024/0200135 A1
(43) Pub. Date: Jun. 20, 2024

(54) Reusable Flow Cells Having Primer Binding Sites Comprising Reactive Sulfur Moieties and Methods of Using the Same, and Reagents for Use Therewith

(71) Applicant: Illumina, Inc., San Diego, CA (US)

(72) Inventors: Weixian Xi, San Diego, CA (US); Brian Mather, San Diego, CA (US); Claudia Battistella, La Jolla, CA (US)

(73) Assignee: Illumina, Inc., San Diego, CA (US)

(21) Appl. No.: 18/538,441

(22) Filed: Dec. 13, 2023

Related U.S. Application Data
(60) Provisional application No. 63/432,153, filed on Dec. 13, 2022.

(51) Int. Cl. C12Q 1/6874; B01L 3/00

(52) U.S. Cl. CPC C12Q 1/6874; B01L 3/502761; B01L 2200/0647; B01L 2300/0654

(57) ABSTRACT

Reusable flow cells for sequencing which exhibit signal intensity retention over numerous use cycles, the active surface of which contains reactive sulfur moieties for reversible primer binding, methods of using such flow cells, reagents therefor, and kits containing the same.

### CROSS-REFERENCE TO RELATED APPLICATIONS

This application claims the benefit of Provisional U.S. Patent Application No. 63/432,153, filed Dec. 13, 2022, the entire disclosure of which is hereby incorporated by reference herein in its entirety.

### BACKGROUND

Flow cells are used in a variety of methods and applications, such as gene sequencing, genotyping, etc. For various analyses such as nucleic acid analysis, the surface of the flow cell may be functionalized with specific surface chemistry, such as primers, polymerases, etc. depending upon the reaction that is to take place. In many instances, the surface chemistry is covalently bound to the flow cell surface. Covalent linking may be desirable to maintain the surface chemistry in the active area of the flow cell throughout various stages of analysis or throughout the lifetime of the flow cell during a variety of uses.

Numerous cycles of use and the associated reactions that take place to functionalize the flow cell surface can degrade the capacity of the flow cell to maintain the surface chemistry necessary for various analyses, and in some cases flow cells can be simply considered a consumable. As in most industries, fewer consumables in a process are desirable.

### BRIEF SUMMARY

The various embodiments of the present invention are directed, in general, to flow cells for sequencing, methods of treating the substrates of flow cells, reagents for such treatment, and kits comprising such flow cells and reagents. Various embodiments described herein provide reusable flow cells having signal intensity retention. Reusable flow cells in accordance with various embodiments herein maintain or retain signal intensity levels in use over numerous cycles of analysis. Various embodiments of the present disclosure provide methods of treating the surface of flow cells to maintain and retain the signal intensity from the flow cell in use over numerous cycles of analysis. Various embodiments of the present disclosure provide reagents for use in such methods.

In various embodiments, at its surface, a flow cell may include a polymeric hydrogel or other surface coating chemistry which includes functional groups that are capable of attaching to primers to be used in nucleic acid sequencing wherein the functional groups include reactive sulfur moieties that are capable of reversible reactions. In various embodiments, at its surface, a flow cell may include reactive sulfur functional groups that are capable of attaching to primers to be used in nucleic acid sequencing, and which are bound to the flow cell surface without the presence of a hydrogel. After a sequencing cycle (i.e., priming, grafting of nucleotides, analysis, data capture, removal of nucleotides, etc.), the primers are removed. In various embodiments of the present disclosure, removal of the primers is carried out by a reaction that reverses the original primer grafting and leaves a reactive sulfur moiety capable of having a new primer grafted thereto. In some instances, primer removal can leave post-sequencing functional groups that are different than the original functional groups that are capable of attaching to the primers. In these instances, the flow cell surface can be contacted with reagents in accordance with various embodiments and using methods in accordance with various embodiments such that the post-sequencing functional groups are converted back into the functional groups that are capable of attaching to the primers, thus maintaining or retaining the signal intensity for the next sequencing cycle. In other instances, primer removal can leave a different reactive sulfur moiety which is capable of reacting with a thiol terminated primer, thus maintaining or retaining the signal intensity for the next sequencing cycle.

One contemplated embodiment includes a flow cell comprising a substrate, the substrate comprising a plurality of primer binding sites, each of the plurality of primer binding sites comprising a reactive sulfur moiety. An additional contemplated embodiment includes a method comprising: providing a flow cell having a substrate comprising a plurality of primer binding sites, each of the plurality of primer binding sites comprising a first reactive sulfur moiety; grafting oligonucleotide primers to a portion of the plurality of primer binding sites; performing a nucleic acid sequence analysis; and contacting the flow cell substrate with a cleaving reagent that removes the oligonucleotide primers such that the substrate comprises a plurality of primer binding sites comprising a second reactive sulfur moiety. An additional embodiment includes a kit comprising: a flow cell having a substrate comprising a plurality of primer binding sites, each of the plurality of primer binding sites comprising a first reactive sulfur moiety; a priming reagent comprising oligonucleotide primers having a terminal group that forms a bond with the primer binding sites; and a cleaving reagent that is capable of removing the oligonucleotide primers such that the substrate comprises a plurality of primer binding sites comprising a second reactive sulfur moiety.

In various contemplated embodiments, the one or more reactive sulfur moieties can be selected from the group consisting of thiols, thioesters and disulfides. In various embodiments, the one or more reactive sulfur moieties may comprise a pyridyl disulfide moiety, for example, as represented herein by the formula (Ia):

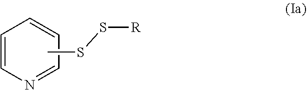

wherein R represents a moiety or functional group on the surface of a flow cell.

In various contemplated embodiments, the one or more reactive sulfur moieties may comprise a thioester moiety, for example, as represented herein by the formula (III):

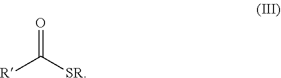

wherein R′ represents a moiety or functional group on the surface of a flow cell and R represents an alkyl or aryl group, which may be further substituted or functionalized so long as the atom of R bound to the sulfur is a carbon.

Another contemplated embodiment includes a method comprising: providing a flow cell having a substrate; and contacting the substrate with a reagent comprising a polymer having a plurality of pendant pyridyl disulfide moieties. Yet another contemplated embodiment includes a reagent comprising a reactive sulfur terminated primer in a carrier fluid.

Other aspects, features and advantages will be apparent from the following disclosure, including the detailed description, preferred embodiments, and the appended claims.

### BRIEF DESCRIPTION OF THE DRAWINGS

The foregoing summary, as well as the following detailed description of illustrated embodiments, will be better understood when read in conjunction with the appended drawings. For purposes of illustration, there are shown in the drawings a number of example embodiments. It should be understood, however, that the disclosure is not limited to the precise arrangements and instrumentalities shown. In the drawings, like reference numerals correspond to similar, though perhaps not identical, components. For the sake of brevity, reference numerals or features having a previously described function may or may not be described in connection with other drawings in which they appear.

FIG. 1A is a top view of a reference flow cell suitable for use in accordance with various embodiments herein;

FIG. 1B through FIG. 1D are enlarged, and partially cutaway views of different examples of a flow channel of the flow cell;

FIG. 2 shows an example of a method in accordance with an embodiment herein;

FIG. 3 depicts a two-step mechanism of native chemical ligation;

FIG. 4 depicts a mechanism for reversing native chemical ligation including an N-to-S acyl shift;

FIG. 5 shows an example of a method in accordance with another embodiment herein;

FIG. 6 shows an example of a method in accordance with another embodiment herein;

FIG. 7A shows an example of reversible oligonucleotide grafting with pyridyl disulfide;

FIG. 7B shows an example of reversible polymer grafting with pyridyl disulfide; and

FIG. 8 illustrates an example of a cross-linking reaction (e.g., a reversible cross-linking reaction) using PDS.

### DETAILED DESCRIPTION

It is to be understood that terms used herein will take on their ordinary meaning in the relevant art unless specified otherwise. Several terms used herein and the meanings encompassed by those terms are set forth below

As used herein, the singular terms “a” and “the” are synonymous and used interchangeably with “one or more” and “at least one,” unless the language and/or context clearly indicates otherwise. Accordingly, for example, reference to “a compound” or “the compound” herein or in the appended claims can refer to a single compound or more than one compound. Additionally, all numerical values, unless otherwise specifically noted, are understood to be modified by the word “about.” The terms comprising, including, containing and various forms of these terms are synonymous with each other and are meant to be equally broad.

The terms first, second, etc. also are not meant to imply a specific orientation or order, but rather are used to distinguish one component from another. It is to be understood that the ranges provided herein include the stated range and any value or sub-range within the stated range, as if such values or subranges were explicitly recited. For example, a range of about 400 nm to about 1 μm (1000 nm), should be interpreted to include not only the explicitly recited limits of about 400 nm to about 1 μm, but also to include individual values, such as about 708 nm, about 945.5 nm, etc., and sub-ranges, such as from about 425 nm to about 825 nm, from about 550 nm to about 940 nm, etc. Furthermore, when “about” and/or “substantially” are/is utilized to describe a value, they are meant to encompass minor variations (up to +/−10%) from the stated value

For simplicity and clarity of illustration, elements in the figures are not necessarily to scale, and the same reference numbers in different figures denote the same elements.

Certain terminology is used in the following description for convenience only and is not limiting. The words “right”, “left”, “lower”, and “upper” designate directions in the drawing to which reference is made and are used herein to describe the flow cell and/or the various components of the flow cell. It is to be understood that these directional terms are not meant to imply a specific orientation, but are used to designate relative orientation between components. The use of directional terms should not be interpreted to limit the examples disclosed herein to any specific orientation(s). The words “inwardly” and “outwardly” refer direction toward and away from, respectively, the geometric center of the object described and designated parts thereof. The terminology includes the words above specifically mentioned, derivatives thereof and words of similar import.

An “acrylamide” is a functional group with the structure

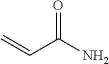

where each H may alternatively be an alkyl, an alkylamino, an alkylamido, an alkylthio, an aryl, a glycol, and optionally substituted variants thereof.

As used herein, “alkyl” refers to a straight or branched hydrocarbon chain that is fully saturated (i.e., contains no double or triple bonds). The alkyl group may have 1 to 20 carbon atoms. Example alkyl groups include methyl, ethyl, propyl, isopropyl, butyl, isobutyl, tertiary butyl, pentyl, hexyl, and the like. As an example, the designation “C1-C6 alkyl” indicates that there are one to six carbon atoms in the alkyl chain, i.e., the alkyl chain is selected from the group consisting of methyl, ethyl, propyl, iso-propyl, n-butyl, isobutyl, sec-butyl, t-butyl, pentyl, and hexyl.

As used herein, “alkylamino” refers to an alkyl group in which one or more of the hydrogen atoms are replaced by an amino group, where the amino group refers to an —NRₐR_{b} group, where Rₐ and R_{b} are each independently selected from a C1-C6 alkyl, C2-C6 alkenyl, C2-C6 alkynyl, C3-C7 carbocycle, C6-C10 aryl, a 5-10 membered heteroaryl, and a 5-10 membered heterocycle.

As used herein, “alkylamido” refers to an alkyl group in which one or more of the hydrogen atoms are replaced by a C-amido group or an N-amido group. A “Camido” group refers to a “—C(═O)N(RₐR_{b})” group in which Rₐ and R_{b} can independently be selected from the group consisting of alkyl, alkenyl, alkynyl, cycloalkyl, cycloalkenyl, cycloalkynyl, aryl, heteroaryl, heteroalicycle, aralkyl, or (heteroalicyclic)alkyl. An “N-amido” group refers to a “RC(═O)N(Rₐ)—” group in which R and Rₐ can independently be selected from the group consisting of alkyl, alkenyl, alkynyl, cycloalkyl, cycloalkenyl, cycloalkynyl, aryl, heteroaryl, heteroalicycle, aralkyl, or (heteroalicyclic)alkyl. Any alkylamido may be substituted or unsubstituted.

As used herein, “alkylthio” refers to RS—, in which R is an alkyl. The alkylthio can be substituted or unsubstituted.

As used herein, “alkene” or “alkenyl” or “olefin” refers to a straight or branched hydrocarbon chain containing one or more double bonds. The alkenyl group may have 2 to 20 carbon atoms. Example alkenyl groups include ethenyl, propenyl, butenyl, pentenyl, hexenyl, and the like.

As used herein, “alkyne” or “alkynyl” refers to a straight or branched hydrocarbon chain containing one or more triple bonds. The alkynyl group may have 2 to 20 carbon atoms.

An “allyl” refers to the unsaturated hydrocarbon radical —CH═CHCH₂.

As used herein, “aralkyl” and “aryl(alkyl)” refer to an aryl group connected, as a substituent, via a lower alkylene group. The lower alkylene and aryl group of an aralkyl may be substituted or unsubstituted. Examples include but are not limited to benzyl, 2-phenylalkyl, 3-phenylalkyl, and naphthylalkyl.

The term “aryl” refers to an aromatic ring or ring system (i.e., two or more fused rings that share two adjacent carbon atoms) containing only carbon in the ring backbone. When the aryl is a ring system, every ring in the system is aromatic. The aryl group may have 6 to 18 carbon atoms. Examples of aryl groups include phenyl, naphthyl, azulenyl, and anthracenyl. Any aryl may be a heteroaryl, with at least one heteroatom, that is, an element other than carbon (e.g., nitrogen, oxygen, sulfur, etc.), in ring backbone.

As used herein, the term “attached” refers to the state of two things being joined, fastened, adhered, connected or bound to each other, either directly or indirectly. For example, a nucleic acid can be attached to a functionalized polymer by a covalent or non-covalent bond. A covalent bond is characterized by the sharing of pairs of electrons between atoms. A non-covalent bond is a physical bond that does not involve the sharing of pairs of electrons and can include, for example, hydrogen bonds, ionic bonds, van der Waals forces, hydrophilic interactions and hydrophobic interactions.

An “azide” or “azido” functional group refers to —N₃.

As used herein, “carbocycle” means a non-aromatic cyclic ring or ring system containing only carbon atoms in the ring system backbone. When the carbocycle is a ring system, two or more rings may be joined together in a fused, bridged or spiro-connected fashion. Carbocycles may have any degree of saturation, provided that at least one ring in a ring system is not aromatic. Thus, carbocycles include cycloalkyls, cycloalkenyls, and cycloalkynyls. The carbocycle group may have 3 to 20 carbon atoms. Examples of carbocycle rings include cyclopropyl, cyclobutyl, cyclopentyl, cyclohexyl, cyclohexenyl, 2,3-dihydro-indene, bicyclo[2.2.2]octanyl, adamantyl, and spiro[4.4]nonanyl. Any of the carbocycles may be heterocycles, with at least one heteroatom in ring backbone.

As used herein, “cycloalkyl” refers to a completely saturated (no double or triple bonds) mono- or multi-cyclic hydrocarbon ring system. When composed of two or more rings, the rings may be joined together in a fused fashion. Cycloalkyl groups can contain 3 to 10 atoms in the ring(s). In some examples, cycloalkyl groups can contain 3 to 8 atoms in the ring(s). A cycloalkyl group may be unsubstituted or substituted. Example cycloalkyl groups include cyclopropyl, cyclobutyl, cyclopentyl, cyclohexyl, cycloheptyl, and cyclooctyl.

As used herein, “cycloalkenyl” or “cycloalkene” means a carbocycle ring or ring system having at least one double bond, wherein no ring in the ring system is aromatic. Examples include cyclohexenyl or cyclohexene and norbornenyl or norbornene.

As used herein, “cycloalkynyl” or “cycloalkyne” means a carbocycle ring or ring system having at least one triple bond, wherein no ring in the ring system is aromatic. An example is cyclooctyne. Another example is bicyclononyne. Still another example is dibenzocyclooctyne (DBCO).

The term “depositing,” as used herein, refers to any suitable application technique, which may be manual or automated, and, in some instances, results in modification of the surface properties. Generally, depositing may be performed using vapor deposition techniques, coating techniques, grafting techniques, or the like. Some specific examples include chemical vapor deposition (CVD), spray coating (e.g., ultrasonic spray coating), spin coating, dunk or dip coating, doctor blade coating, puddle dispensing, flow through coating, aerosol printing, screen printing, microcontact printing, inkjet printing, or the like.

As used herein, the term “depression” refers to a discrete concave feature in a substrate or a patterned material having a surface opening that is at least partially surrounded by interstitial region(s) of the substrate or the patterned material. Depressions can have any of a variety of shapes at their opening in a surface including, as examples, round, elliptical, square, polygonal, star shaped (with any number of vertices), etc. The cross-section of a depression taken orthogonally with the surface can be curved, square, polygonal, hyperbolic, conical, angular, etc. As examples, the depression can be a well or two interconnected wells. The depression may also have more complex architectures, such as ridges, step features, etc.

The term “each,” when used in reference to a collection of items, is intended to identify an individual item in the collection, but does not necessarily refer to every item in the collection. Exceptions can occur if explicit disclosure or context clearly dictates otherwise.

As used herein, the term “flow cell” is intended to mean a vessel having a flow channel that is in fluid communication with at least one unmodified surface or at least one surface modified with a first member of a transition metal complex binding pair. The unmodified or modified surface is capable of attaching surface chemistry that to be used in during a nucleic acid analysis, and is capable of releasing the surface chemistry either electrochemically or upon exposure to visible light. The flow cell also includes an inlet for delivering reagent(s) to the flow channel and an outlet for removing reagent(s) from the flow channel. The flow cell enables the detection of the reactions involving the surface chemistry. For example, the flow cell may include one or more transparent surfaces, which allow for the optical detection of arrays, optically labeled molecules, or the like within the flow channel.

As used herein, a “flow channel” or “channel” may be an area defined between two bonded components, which can selectively receive a liquid sample. In some examples, the flow channel may be defined between a patterned or nonpatterned structure and a lid. In other examples, the flow channel may be defined between two patterned or non-patterned structures that are bonded together.

As used herein, “heteroalicyclic” or “heteroalicycle” refers to three-, four-, five-, six-, seven-, eight-, nine-, ten-, up to 18-membered monocyclic, bicyclic, and tricyclic ring system wherein carbon atoms together with from 1 to 5 heteroatoms constitute said ring system. A heteroalicyclic ring system may optionally contain one or more unsaturated bonds situated in such a way, however, that a fully delocalized pielectron system does not occur throughout all the rings. The heteroatoms are independently selected from oxygen, sulfur, and nitrogen. A heteroalicyclic ring system may further contain one or more carbonyl or thiocarbonyl functionalities, so as to make the definition include oxo-systems and thio-systems such as lactams, lactones, cyclic imides, cyclic thioimides, and cyclic carbamates. The rings may be joined together in a fused fashion. Additionally, any nitrogens in a heteroalicyclic may be quaternized. Heteroalicycle or heteroalicyclic groups may be unsubstituted or substituted. Examples of such “heteroalicyclic” or “heteroalicycle” groups include 1,3dioxin, 1,3-dioxane, 1,4-dioxane, 1,2-dioxolane, 1,3-dioxolane, 1,4-dioxolane, 1,3-oxathiane, 1,4-oxathiin, 1,3-oxathiolane, 1,3-dithiole, 1,3-dithiolane, 1,4-oxathiane, tetrahydro-1,4-thiazine, 2H-1,2-oxazine, maleimide, succinimide, barbituric acid, thiobarbituric acid, dioxopiperazine, hydantoin, dihydrouracil, trioxane, hexahydro1,3,5-triazine, imidazoline, imidazolidine, isoxazoline, isoxazolidine, oxazoline, oxazolidine, oxazolidinone, thiazoline, thiazolidine, morpholine, oxirane, piperidine N-oxide, piperidine, piperazine, pyrrolidine, pyrrolidone, pyrrolidione, 4-piperidone, pyrazoline, pyrazolidine, 2-oxopyrrolidine, tetrahydropyran, 4H-pyran, tetrahydrothiopyran, thiamorpholine, thiamorpholine sulfoxide, thiamorpholine sulfone, and their benzo-fused analogs (e.g., benzimidazolidinone, tetrahydroquinoline, 3,4-methylenedioxyphenyl).

A “(heteroalicyclic)alkyl” refers to a heterocyclic or a heteroalicyclic group connected, as a substituent, via a lower alkylene group. The lower alkylene and heterocycle or a heterocycle of a (heteroalicyclic)alkyl may be substituted or unsubstituted. Examples include tetrahydro-2H-pyran-4-yl)methyl, (piperidin-4yl)ethyl, (piperidin-4-yl)propyl, (tetrahydro-2H-thiopyran-4-yl)methyl, and (1,3thiazinan-4-yl)methyl.

As used herein, “hydroxy” or “hydroxyl” refers to an —OH group.

As used herein, the term “glycol” refers to the end group —(CH₂)ₙOH, where n ranges from 2 to 10. As specific examples, the glycol may be an ethylene glycol end group —CH₂CH₂OH, a propylene glycol end group —CH₂CH₂CH₂OH, or a butylene glycol end group —CH₂CH₂CH₂CH₂OH.

As used herein, the term “interstitial region” refers to an area, e.g., of a substrate, patterned resin, or other support that separates depressions or protrusions. For example, an interstitial region can separate one depression of an array from another depression of the array, or one protrusion of an array from another protrusion of an array. The two depressions or protrusions that are separated from each other can be discrete, i.e., lacking physical contact with each other. In many examples, the interstitial region is continuous whereas the depressions or protrusions are discrete, for example, as is the case for a plurality of depressions defined in an otherwise continuous surface. In other examples, the interstitial regions and the features (e.g., depressions or protrusions) are discrete, for example, as is the case for a plurality of trenches separated by respective interstitial regions. The separation provided by an interstitial region can be partial or full separation. Interstitial regions may have a surface material that differs from the surface material of the depressions or protrusions. For example, the depression or protrusion surface can include the polymeric hydrogel, while the interstitial regions are free of the polymeric hydrogel.

As used herein, a “nucleotide” includes a nitrogen containing heterocyclic base, a sugar, and one or more phosphate groups. Nucleotides are monomeric units of a nucleic acid sequence. In ribonucleic acids (RNA), the sugar is a ribose, and in deoxyribonucleic acids (DNA), the sugar is a deoxyribose, i.e. a sugar lacking a hydroxyl group that is present at the 2′ position in ribose. The nitrogen containing heterocyclic base (i.e., nucleobase) can be a purine base or a pyrimidine base. Purine bases include adenine (A) and guanine (G), and modified derivatives or analogs thereof. Pyrimidine bases include cytosine (C), thymine (T), and uracil (U), and modified derivatives or analogs thereof. The C-1 atom of deoxyribose is bonded to NI of a pyrimidine or N-9 of a purine. A nucleic acid analog may have any of the phosphate backbone, the sugar, or the nucleobase altered. Examples of nucleic acid analogs include, for example, universal bases or phosphate-sugar backbone analogs, such as peptide nucleic acid (PNA).

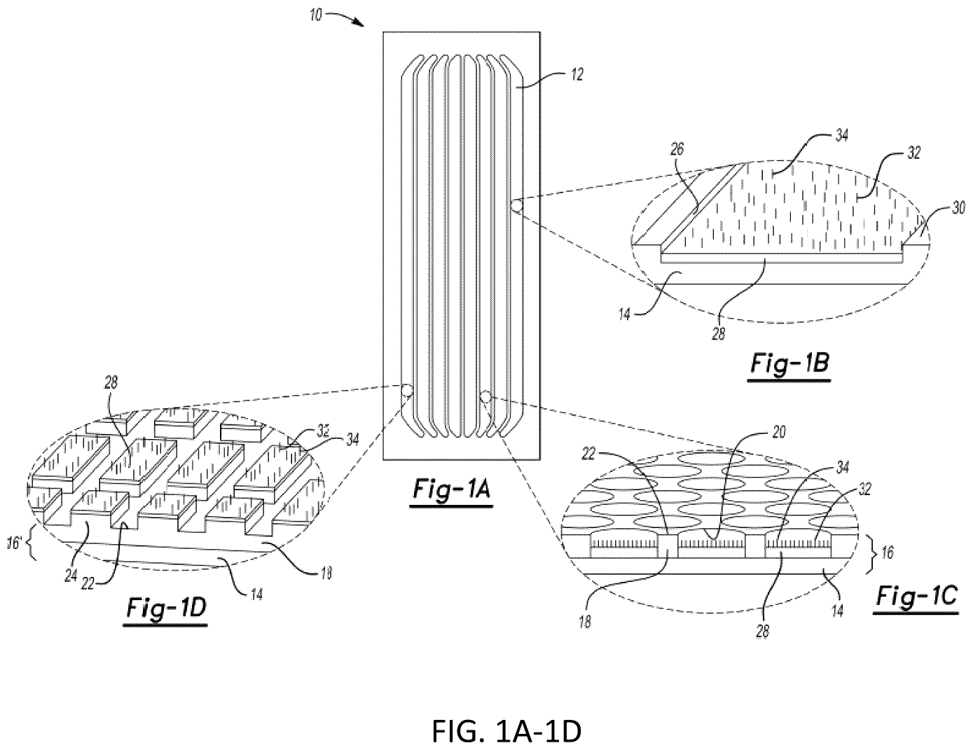

In some examples, the term “over” may mean that one component or material is positioned directly on another component or material. When one is directly on another, the two are in contact with each other. In FIG. 1B, the polymeric hydrogel 28 is applied over the single layer base support 14 so that it is directly on and in contact with the single layer based support 14.

In other examples, the term “over” may mean that one component or material is positioned indirectly on another component or material. By indirectly on, it is meant that a gap or an additional component or material may be positioned between the two components or materials. In FIG. 1D, the polymeric hydrogel 28 is positioned over the base support 14 of the multi-layered structure 16′ such that the two are in indirect contact. More specifically, the layer 18 is positioned between the polymeric hydrogel 28 and the base support 14.

As used herein, the term “primer” is defined as a single stranded nucleic acid sequence (e.g., single strand DNA). Some primers are part of a primer set, which serve as a starting point for template amplification and cluster generation. Other primers, referred to herein as sequencing primers, serve as a starting point for DNA synthesis. The 5′ terminus of a primer set may be modified to allow a coupling reaction with a functional group of one of the orthogonal polymers. The primer length can be any number of bases long and can include a variety of non-natural nucleotides. In an example, the sequencing primer is a short strand, ranging from 10 to 60 bases, or from 20 to 40 bases.

As used herein, the term “pyridyl disulfide” refers to a moiety having the structure of general formula (Ia):

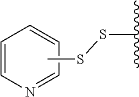

wherein the disulfide may be bound to the pyridine ring at any carbon. For example, the nitrogen of the pyridyl ring may be located at the 2-position, such as the structure of general formula (Ib):

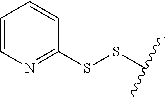

As used herein, the term “dipyridyl disulfide” refers to a compound having the general formula (Ic):

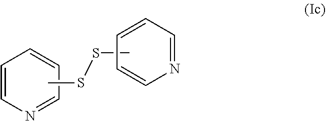

wherein each pyridine ring can be bound to the disulfide bridge at any carbon atom. For example, various embodiments may include a dipyridyl disulfide having the general formula (Id):

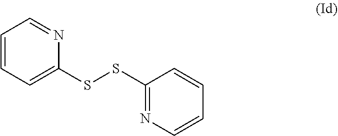

herein referred to as “2,2′-dipyridyl disulfide” or “aldrithiol,” or a dipyridyl disulfide of the general formula (Ie):

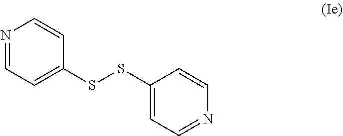

herein referred to as “4,4′-dipyridyl disulfide,” or mixtures of the two disulfides, and/or additional disulfides.

The term “substrate” refers to a structure upon which various components of the flow cell (e.g., polymeric hydrogel, primers, etc.) may be added. The substrate may be a wafer, a panel, a rectangular sheet, a die, or any other suitable configuration. The substrate is generally rigid and is insoluble in an aqueous liquid. The substrate may be a single layer structure, or a multi-layered structure (e.g., including a support and a patterned material on the support). Examples of suitable substrates will be described further herein.

As used herein, the terms “tetrazine” and “tetrazinyl” refer to six-membered heteroaryl group comprising four nitrogen atoms. Tetrazine can be optionally substituted.

As used herein, the term thioester refers to a functional group having the general formula (III):

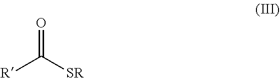

wherein R′ represents a moiety or functional group on the surface of a flow cell and R represents an alkyl or aryl group which group may bear one or more substituents or be further functionalized, so long as the R group atom bound to the sulfur is a carbon. For example, in various embodiments, R may represent —CH₂CH₂COOH.

Flow cells suitable for use in accordance with various method embodiments herein can include any of the following suitable structures.

One example of a flow cell 10 having a suitable architecture for use in all embodiments is shown in FIG. 1A from a top view. The flow cell 10 may include two patterned or non-patterned structures bonded together, or one patterned or non-patterned structure bonded to a lid.

The patterned structures, the non-patterned structures, or the patterned or non-patterned structure and the lid) may be attached to one another through a spacer layer (not shown). The spacer layer may be any material that will seal portions of the patterned or non-patterned structures together or portions of the patterned or nonpatterned structure and the lid. As examples, the spacer layer may be an adhesive, a radiation-absorbing material that aids in bonding, or the like. In some examples, the spacer layer is the radiation-absorbing material, e.g., KAPTON® black. The patterned or non-patterned structures or the patterned structure and the lid may be bonded using any suitable technique, such as laser bonding, diffusion bonding, anodic bonding, eutectic bonding, plasma activation bonding, glass frit bonding, or other methods known in the art.

Between the two patterned or non-patterned structures or the one patterned or non-patterned structure and the lid is a flow channel 12. The example shown in FIG. 1A includes eight flow channels 12. While eight flow channels 12 are shown, it is to be understood that any number of flow channels 12 may be included in the flow cell 10 (e.g., a single flow channel 12, four flow channels 12, etc.). Each flow channel 12 may be isolated from another flow channel 12 so that fluid introduced into a flow channel 12 does not flow into adjacent flow channel(s) 12. Some examples of the fluids introduced into the flow channel 12 may introduce reaction components (e.g., cleaving fluids, DNA sample, polymerases, sequencing primers, nucleotides, etc.), washing solutions, deblocking agents, etc.

The flow channel 12 may have any desirable shape. In an example, the flow channel 12 has a substantially rectangular configuration. The length of the flow channel 12 depends, in part, upon the size of the substrate upon which the patterned or non-patterned structure is formed. The width of the flow channel 12 depends, in part, upon the size of the substrate upon which the patterned or non-patterned structure is formed, the desired number of flow channels 12, the desired space between adjacent channels 12, and the desired space at a perimeter of the patterned or non-patterned structure.

The depth of the flow channel 12 can be as small as a monolayer thick when microcontact, aerosol, or inkjet printing is used to deposit a separate material that defines the flow channel 12 walls. For other examples, the depth of the flow channel 12 can be about 1 μm, about 10 μm, about 50 μm, about 100 μm, or more. In an example, the depth may range from about 10 μm to about 100 μm. In another example, the depth may range from about 10 μm to about 30 μm. In still another example, the depth is about 5 μm or less. It is to be understood that the depth of the flow channel 12 may be greater than, less than or between the values specified above.

Each flow channel 12 is in fluid communication with an inlet and an outlet (not shown). The inlet and outlet of each flow channel 12 may be positioned at opposed ends of the flow cell 10. The inlets and outlets of the respective flow channels 12 may alternatively be positioned anywhere along the length and width of the flow channel 12 that enables desirable fluid flow.

The inlet allows fluids to be introduced into the flow channel 12, and the outlet allows fluid to be extracted from the flow channel 12. Each of the inlets and outlets is fluidly connected to a fluidic control system (including, e.g., reservoirs, pumps, valves, waste containers, and the like) which controls fluid introduction and expulsion.

FIG. 1B, FIG. 1C, and FIG. 1D depict different examples of the architecture within the flow channel 12.

Each of the architectures includes a substrate, such as a single layer base support 14 (as shown in FIG. 1B), or a multi-layered structure 16, 16′ (as shown in FIG. 1C and FIG. 1D, respectively).

Examples of suitable single layer base supports 14 include epoxy siloxane, glass, modified or functionalized glass, plastics (including acrylics, polystyrene and copolymers of styrene and other materials, polypropylene, polyethylene, polybutylene, polyurethanes, polytetrafluoroethylene (such as TEFLON® from Chemours), cyclic olefins/cyclo-olefin polymers (COP) (such as ZEONOR® from Zeon), polyimides, etc.), nylon (polyamides), ceramics/ceramic oxides, silica, fused silica, or silica-based materials, aluminum silicate, silicon and modified silicon (e.g., boron doped p+ silicon), silicon nitride (Si₃N₄), silicon oxide (SiO₂), tantalum pentoxide (Ta₂O₅) or other tantalum oxide(s) (TaOₓ), hafnium oxide (HfO₂), carbon, metals, inorganic glasses, or the like.

Examples of the multi-layered structure 16, 16′ include the base support 14 and at least one other layer 18 thereon, as shown in FIG. 1C and FIG. 1D.

Some examples of the multi-layered structure 16, 16′ include glass or silicon as the base support 14, with a coating layer (e.g., layer 18) of tantalum oxide (e.g., tantalum pentoxide or another tantalum oxide(s) (TaOₓ)) or another ceramic oxide at the surface.

Other examples of the multi-layered structure 16, 16′ include the base support 14 (e.g., glass, silicon, tantalum pentoxide, or any of the other base support 14 materials) and a patterned resin as the other layer 18. It is to be understood that any material that can be selectively deposited, or deposited and patterned to form depressions 20 and interstitial regions 22 (FIG. 1C) or protrusions 24 and interstitial 20 regions 22 (FIG. 1D) may be used for the patterned resin.

As one example of the patterned resin, an inorganic oxide may be selectively applied to the base support 14 via vapor deposition, aerosol printing, or inkjet printing. Examples of suitable inorganic oxides include tantalum oxide (e.g., Ta₂O₅), aluminum oxide (e.g., Al₂O₃), silicon oxide (e.g., SiO₂), hafnium oxide (e.g., 25 HfO₂), etc.

As another example of the patterned resin, a polymeric resin may be applied to the base support 14 and then patterned. Suitable deposition techniques include chemical vapor deposition, dip coating, dunk coating, spin coating, spray coating, puddle dispensing, ultrasonic spray coating, doctor blade coating, aerosol printing, screen printing, microcontact printing, etc. Suitable patterning techniques include photolithography, nanoimprint lithography (NIL), stamping techniques, embossing techniques, molding techniques, microetching techniques, etc. Some examples of suitable resins include a polyhedral oligomeric silsesquioxane resin (POSS)-based resin, a non-POSS epoxy resin, a poly(ethylene glycol) resin, a polyether resin (e.g., ring opened epoxies), an acrylic resin, an acrylate resin, a methacrylate resin, an amorphous fluoropolymer resin (e.g., CYTOP® from Bellex), and combinations thereof.

As used herein, the term “polyhedral oligomeric silsesquioxane” (POSS) refers to a chemical composition that is a hybrid intermediate (e.g., RSiO_{1.5}) between that of silica (SiO₂) and silicone (R₂SiO). An example of POSS may be that described in Kehagias et al., Microelectronic Engineering 86 (2009), pp. 776-778, which is incorporated by reference in its entirety. In an example, the composition is an organosilicon compound with the chemical formula [RSiO_{3/2}]ₙ, where the R groups can be the same or different. Example R groups for POSS include epoxy, azide/azido, a thiol, a poly(ethylene glycol), a norbornene, a tetrazine, acrylates, and/or methacrylates, or further, for example, alkyl, aryl, alkoxy, and/or haloalkyl groups.

In an example, the single base support 14 (whether used singly or as part of the multi-layered structure 16, 16′) may be a circular sheet, a panel, a wafer, a die, etc. having a diameter ranging from about 2 mm to about 300 mm, e.g., from about 200 mm to about 300 mm, or may be a rectangular sheet, panel, wafer, die etc. having its largest dimension up to about 10 feet (˜3 meters). For example, a die may have a width ranging from about 0.1 mm to about 10 mm. While example dimensions have been provided, it is to be understood that a single base support 14 with any suitable dimensions may be used.

The architecture shown in FIG. 1B is a non-patterned structure. The substrate of the non-patterned structure may be the single layer base support 14. In this example, the single layer base support 14 has a lane 26 surrounded by edge regions 30. The lane 26 provides a designated area for the polymeric hydrogel 28. The edge regions 30 provide bonding regions where two non-patterned structures can be attached to one another or where one non-patterned structure can be attached to a lid. As such, in this example, the surface of the flow cell is non-patterned, and the polymeric hydrogel 28 is positioned within the lane 26 of the non-patterned surface.

The polymeric hydrogel 28 may be any gel material that can swell when liquid is taken up and can contract when liquid is removed, e.g., by drying. In an example of a flow cell suitable for use in various method embodiments described herein, including methods involving treatment with sequencing reagents, the polymeric hydrogel 28 can include an acrylamide copolymer having terminal azide functionalities, such as, for example, poly(N-(5-azidoacetamidylpentyl)acrylamide-co-acrylamide, PAZAM, or norbornene silane polymers, etc. In embodiments where the flow cell surface comprises such polymeric materials, a plurality of primer binding sites having reactive sulfur moieties can be grafted to the flow cell surface using a polymer having both pendant reactive sulfur moieties and pendant complimentary click chemistry moieties that attach to azides, amines, and cyclic alkenes such as norbornene.

In various embodiments, the polymeric hydrogel may be functionalized to include pendant thioester and/or thiol moieties. For example, a dibenzocyclooctyne (DBCO)-thioester can be coupled to a PAZAM surface. In another example, polymer resins having pendant thioester and/or thiol functional groups can be formed on the surface of the flow cell.

To introduce the polymeric hydrogel 28 into the lane 26, a mixture of the polymeric hydrogel 28 may be generated and then applied to the single layer base support 14. In one example, the polymeric hydrogel 28 may be present in a mixture (e.g., with water or with ethanol and water). The mixture may then be applied to the respective substrate surfaces (including in the lane 26) using spin coating, or dipping or dip coating, or flow of the material under positive or negative pressure, or another suitable technique. These types of techniques blanketly deposit the polymeric hydrogel 28 in the lane 26 and on the edge regions 30. Other selective deposition techniques (e.g., involving a mask, controlled printing techniques, etc.) may be used to specifically deposit the polymeric hydrogel 28 in the lane 26 and not on the edge regions 30.

In some examples, the surface of the single layer base support 14 (including in the lane 26) may be activated, and then the mixture (including the polymeric hydrogel 28) may be applied thereto. In one example, a silane or silane derivative (e.g., norbornene silane) may be deposited on the surface of the single layer base support 14 using vapor deposition, spin coating, or other deposition methods. In another example, the substrate surface may be exposed to plasma ashing to generate surface activating agent(s) (e.g., —OH groups) that can adhere to the polymeric hydrogel 28.

Depending upon the chemistry of the polymeric hydrogel 28, the applied mixture may be exposed to a curing process. In an example, curing may take place at a temperature ranging from room temperature (e.g., about 25° C.) to about 95° C. for a time ranging from about 1 millisecond to about several days.

Polishing may then be performed in order to remove the polymeric hydrogel 28 from the edge regions 30 at the perimeter of the lane 26, while leaving the polymeric hydrogel 28 on the surface in the lane 26 at least substantially intact.

The architecture shown in FIG. 1C is one example of a patterned structure. The substrate of this patterned structure is the multi-layered structure 16 with depressions 20 defined in the layer 18. The depressions 20 provide a designated area for the polymeric hydrogel 28. In this example, the surface of the flow cell 10 is patterned with depressions 20 separated by interstitial regions 22, and the polymeric hydrogel 28 is positioned within each depression 20 of the patterned surface.

Many different layouts of the depressions 20 may be envisaged, including regular, repeating, and non-regular patterns. In an example, the depressions 20 are disposed in a hexagonal grid for close packing and improved density. Other layouts may include, for example, rectangular layouts, triangular layouts, and so forth. In some examples, the layout or pattern can be an x-y format in rows and columns. In some other examples, the layout or pattern can be a repeating arrangement of the depressions 20 and the interstitial regions 22. In still other examples, the layout or 10 pattern can be a random arrangement of the depressions 20 and the interstitial regions 22.

The layout or pattern may be characterized with respect to the density (number) of the depressions 20 in a defined area. For example, the depressions 20 may be present at a density of approximately 2 million per mm². The density may be tuned to different densities including, for example, a density of about 100 per mm², about 1,000 per mm², about 0.1 million per mm², about 1 million per mm², about 2 million per mm², about 5 million per mm², about 10 million per mm², about 50 million per mm², or more, or less. It is to be further understood that the density can be between one of the lower values and one of the upper values selected from the ranges above, or that other densities (outside of the given ranges) may be used. As examples, a high density array may be characterized as having depressions 20 separated by less than about 100 nm, a medium density array may be characterized as having the depressions 20 separated by about 400 nm to about 1 μm, and a low density array may be characterized as having the depressions 20 separated by greater than about 1 μm.

The layout or pattern of the depressions 20 may also or alternatively be characterized in terms of the average pitch, or the spacing from the center of one depression 20 to the center of an adjacent depression 20 (center-to-center spacing) or from the right edge of one depression 20 to the left edge of an adjacent depression 20 (edge-to-edge spacing). The pattern can be regular, such that the coefficient of variation around the average pitch is small, or the pattern can be non-regular in which case the coefficient of variation can be relatively large. In either case, the average pitch can be, for example, about 50 nm, about 0.1 μm, about 0.5 μm, about 1 μm, about 5 μm, about 10 μm, about 100 μm, or more or less. The average pitch for a particular pattern can be between one of the lower values and one of the upper values selected from the ranges above. In an example, the depressions 28A, 28B have a pitch (center-to-center spacing) of about 1.5 μm. While example average pitch values have been provided, it is to be understood that other average pitch values may be used.

The size of each depression 20 may be characterized by its volume, opening area, depth, and/or diameter. For example, the volume can range from about 1×10⁻³ μm³ to about 100 μm³, e.g., about 1×10 2 μm³, about 0.1 μm³, about 1 μm³, about 10 μm³, or more, or less. For another example, the opening area can range from about 1×10 3 μm² to about 100 μm², e.g., about 1×10 2 μm², about 0.1 μm², about 1 μm², at least about 10 μm², or more, or less. For still another example, the depth can range from about 0.1 μm to about 100 μm, e.g., about 0.5 μm, about 1 μm, about 10 μm, or more, or less. For yet another example, the diameter or length and width can range from about 0.1 μm to about 100 μm, e.g., about 0.5 μm, about 1 μm, about 10 μm, or more, or less.

Any example of the polymeric hydrogel 28 disclosed herein may be used in the architecture shown in FIG. 1C.

To introduce the polymeric hydrogel 28 into the depressions 20, a mixture of the polymeric hydrogel 28 may be generated and then applied to the multi-layered structure 16. In one example, the polymeric hydrogel 28 may be present in a mixture (e.g., with water or with ethanol and water). The mixture may then be applied to the respective substrate surfaces (including in the lane 26) using spin coating, or dipping or dip coating, or flow of the material under positive or negative pressure, or another suitable technique. These types of techniques blanketly deposit the polymeric hydrogel 28 in the depressions and on the interstitial regions 22. Other selective deposition techniques (e.g., involving a mask, controlled printing techniques, etc.) may be used to specifically deposit the polymeric hydrogel 28 in the depressions 20 and not on the interstitial regions 22.

In some examples, the surface of the layer 18 (including the depressions 20) may be activated, and then the mixture (including the polymeric hydrogel 28) may be applied thereto. In one example, a silane or silane derivative (e.g., norbornene silane) may be deposited on the surface of the layer 18 using vapor deposition, spin coating, or other deposition methods. In another example, the layer 18 may be exposed to plasma ashing to generate surface-activating agent(s) (e.g., —OH groups) that can adhere to the polymeric hydrogel 28.

Depending upon the chemistry of the polymeric hydrogel 28, the applied mixture may be exposed to a curing process. In an example, curing may take place at a temperature ranging from room temperature (e.g., about 25° C.) to about 95° C. for a time ranging from about 1 millisecond to about several days.

Polishing may then be performed in order to remove the polymeric hydrogel 28 from the interstitial regions 22, while leaving the polymeric hydrogel 28 on the surface in the depressions 20 at least substantially intact.

The architecture shown in FIG. 1D is another example of a patterned structure. The substrate of this patterned structure is the multi-layered structure 16′ with protrusions 24 defined in the layer 18. The protrusions 24 are three-dimensional structures that extend outward (upward) from an adjacent surface. The protrusions 24 may be generated via etching, photolithography, imprinting, etc. In this example, the surface of the flow cell 10 is patterned with protrusions 24 separated by interstitial regions 22, and the polymeric hydrogel 28 is positioned on each protrusion 24 of the patterned surface.

While any suitable three-dimensional geometry may be used for the protrusion 24, a geometry with an at least substantially flat top surface may be desirable. Example protrusion geometries include a sphere, a cylinder, a cube, polygonal prisms (e.g., rectangular prisms, hexagonal prisms, etc.), or the like.

Many different layouts of the protrusions 24 may be envisaged, including ay of those described herein for the depressions 20. The layout or pattern may be characterized with respect to the density (number) of the protrusions 24 in a defined area. The protrusions 24 may be present at a density of approximately 2 million per mm² or at any of the other examples set forth herein for the depressions 20. The layout or pattern of the protrusions 24 may also or alternatively be characterized in terms of the average pitch, or the spacing from the center of one protrusion 24 to the center of an adjacent protrusion 24 (center-to-center spacing) or from the right edge of one protrusion 24 to the left edge of an adjacent protrusion 24 (edge-to-edge spacing).

The size of each protrusion 24 may be characterized by its surface area. The surface area of the protrusion 28 may range from about 1×10 3 μm² to about 100 μm², e.g., about 1×10 2 μm², about 0.1 μm², about 1 μm², at least about 10 μm², or more, or less.

The height of each protrusion 24 (measured from the interstitial region 22) 10 may range from about 10 nm to about 500 nm).

Any example of the polymeric hydrogel 28 disclosed herein may be used in the architecture shown in FIG. 1D. To introduce the polymeric hydrogel 28 onto the protrusions 24, a mixture of the polymeric hydrogel 28 may be generated and then applied to the protrusions 24. Selective deposition techniques may be used to deposit the polymeric hydrogel 28 on the protrusions 24 and not the interstitial regions 22. A mask may be used to cover the interstitial regions 22 while the polymeric hydrogel is deposited on the protrusions 24.

Each example of the flow cell architecture also includes primers 32, 34. The primers 32, 34 may be introduced to the flow cell 10 and bound to reactive sulfur moieties on the flow cell surface at the outset of a nucleic acid analysis. Several primers 32, 34 are discussed below in reference to the various kits and methods.

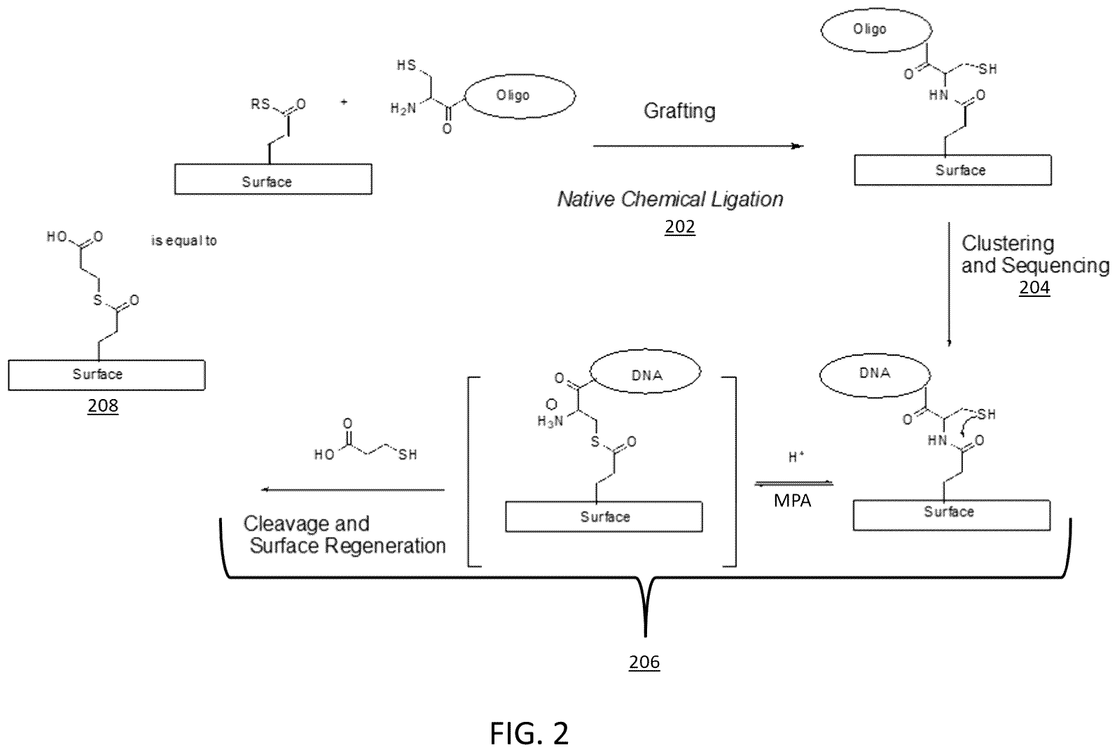

Examples of the flow cell 10 disclosed herein may be used in a variety of methods that provide reactive sulfur moieties for primer binding after a sequencing cycle has been performed, and may be included in a variety of kits with fluids to be used in the methods. The kits and methods will now be described in reference to FIGS. 2-8.

In a first example, a kit in accordance with one embodiment includes: a reusable flow cell that includes at least one surface having a plurality of primer binding sites, each of the plurality of primer binding sites comprising a thioester moiety; a primer fluid including a plurality of cysteine-terminated primers; and a cleaving fluid containing a compound that is reactive with the bond created between a thioester and cysteine, such as, for example 3-mercaptopropionic acid (MPA). This kit may be used in the example method shown in FIG. 2.

In this example kit, the flow cell 10 may be any of the examples described herein in reference to FIG. 1B through FIG. 1D. The surface functionalized with the polymeric hydrogel may be any of the patterned or non-patterned structures described herein, which may include any example of the polymeric hydrogel 28.

This example kit includes the primer fluid. The primer fluid includes a plurality of cysteine-terminated primers, e.g., primers, in a carrier liquid. The cysteine-terminated primers may include forward and reverse amplification primer sequences that are terminated with a cysteine residue for reaction with the sulfur-containing functional group of the polymeric hydrogel. The primers together enable the amplification of a library template having end adapters that are complementary to the two different primers.

As examples, the primer sequences of the cysteine-terminated primers may include P5 and P7 primer sequences; P15 and P7 primer sequences; or any combination of the PA primer sequences, the PB primer sequences, the PC primer sequences, and the PD primer sequences set forth herein.

Examples of P5 and P7 primer sequences are used on the surface of commercial flow cells sold by Illumina Inc. for sequencing, for example, on HISEQ™, HISEQX™, MISEQ™, MISEQDX™, MINISEQ™, NEXTSEQ™, NEXTSEQDX™, NOVASEQ™, ISEQ™, GENOME ANALYZER™, and other instrument platforms.

Primer sequences consistent with the present disclosure may include a cleavage site, such as uracil, 8oxoguanine, allyl-T, etc. at any point in the strand.

Each of the cysteine-terminated primers in the first example kit may also include a polyT sequence. In some examples, the polyT region includes from 2 T bases to 20 T bases. As specific examples, the polyT region may include 3, 4, 5, 6, 7, or 10 T bases.

The cysteine-terminated primers may be included in a carrier liquid in a concentration ranging from about 5 μM to about 10 μM.

The carrier liquid of the primer fluid in the first example kit may be water. A buffer may be added to the carrier liquid for grafting the primers to suitable functional groups of the polymeric hydrogel. The buffer has a pH ranging from 7 to 10, and the buffer used will depend upon the cysteine-terminated primers being used. Examples of buffers include Tris(hydroxymethyl) aminomethane (TRIS) buffers, such as TRIS-HCl or TRIS-EDTA, or sodium sulfate, Tris(hydroxymethyl) aminomethane (CHES) and 3-(Cyclohexylamino)-1-propanesulphonic acid (CAPS).

The first example kit can also include a cleaving fluid. The cleaving fluid is reactive with bond between the surface thioester and the terminal cysteine residue. In general, the cleaving fluid may comprise any thiol-functionalized reagent. For example, the cleaving fluid may contain 3-mercaptopropionic acid, an arylthiol, or 4-mercaptophenylacetic acid, or the like.

A method in accordance with one embodiment which can utilize the first example kit, an example of which is illustrated in FIG. 2, includes: at 202, grafting a plurality of cysteine-terminated primers incorporating a template strand to the thioester moieties of a polymeric hydrogel on a surface of a flow cell; at 204, performing a nucleic acid analysis, including performing steps of clustering (i.e., locally amplifying) the template strand and sequencing of clonal populations; at 206, introducing a cleaving fluid (e.g., MPA) to cleave the grafted plurality of cysteine-terminated primers at the bond between the thioester and cysteine residue, thereby, at 208, leaving a plurality of thioester moieties at the surface of the flow cell.

It is to be understood that in this example of the method, the plurality of cysteine-terminated primers may alternatively be pre-grafted to the flow cell. In these examples, the method would include performing a nucleic acid analysis involving the grafted plurality of primers; introducing the cleaving fluid to cleave the grafted plurality of alkyne-containing primers at the thioester-cysteine bond, thereby leaving a plurality of thioester functional groups at the surface of the flow cell.

If the cysteine-terminated primers are not pre-grafted to the polymeric hydrogel of the flow cell, the method involves grafting the primers to at least some of the thioester functional groups of the polymeric hydrogel. For grafting, the primer fluid of the first example kit is introduced into the flow cell. The primer fluid may be introduced using flow through deposition. Grafting may be performed at a variety of temperatures, for example, at room temperature, or a temperature ranging from about 55° C. to about 65° ° C. for a time ranging from about 20 minutes to about 60 minutes. In one example, grafting is performed at 60° ° C. for about 30 minutes. During grafting, the cysteine-terminated primers attach to at least some of the thioester groups of the polymeric hydrogel and have no affinity for the interstitial regions 22 or edge portions 30 of the flow cell 10.

While the native chemical ligation reaction between a thioester and cysteine-terminated primer is generally near quantitative, there may be residual thioesters on the surface of the flow cell, and such residual thioester moieties can be capped prior to nucleic acid analysis, for example, by hydrolysis to carboxylic acids with a 0.1N NaOH solution.

The nucleic acid analysis may then be performed. In one example, the nucleic acid analysis involves introducing a sample including a plurality of template nucleic acid strands into the flow cell, whereby at least some of the plurality of template nucleic acid strands respectively hybridize to the primer sequence of at least some of the grafted plurality of primers; and performing sequencing-by-synthesis. Sequencing-by-synthesis involves amplification of the template nucleic acid strands and sequencing of the amplified template nucleic acid strands.

The sample including a plurality of template nucleic acid strands (i.e., library templates) may first be prepared from any nucleic acid sample (e.g., a DNA sample or an RNA sample). The DNA nucleic acid sample may be fragmented into single-stranded, similarly sized (e.g., <1000 bp) DNA fragments. The RNA nucleic acid sample may be used to synthesize complementary DNA (cDNA), and the cDNA may be fragmented into single-stranded, similarly sized (e.g., <1000 bp) cDNA fragments. During preparation, adapters may be added to the ends of any of the fragments. Through reduced cycle amplification, different motifs may be introduced in the adapters, such as sequencing primer binding sites, indices, and regions that are complementary to the primers on the flow cell surface. The final library templates include the DNA or cDNA fragment and adapters at both ends. The DNA or cDNA fragment represents the portion of the final library template that is to be sequenced.

The sample may be introduced to the flow cell. The template nucleic acid strands hybridize, for example, to one of two types of primers.

Referring again to FIG. 1, amplification of the template nucleic acid strand(s) may be initiated to form a cluster of the template stands across the polymeric hydrogel (e.g., in the lane 26 (FIG. 1B), in each depression 20 (FIG. 1C), or on each protrusion 24 (FIG. 1D)). In one example, amplification involves cluster generation. In one example of cluster generation, the library templates are copied from the hybridized primers by 3′ extension using a high-fidelity DNA polymerase. The original library templates are denatured, leaving the copies immobilized to the polymeric hydrogel. Isothermal bridge amplification or some other form of amplification may be used to amplify the immobilized copies. For example, the copied templates loop over to hybridize to an adjacent, complementary primer, and a polymerase copies the copied templates to form double stranded bridges, which are denatured to form two single stranded strands. These two strands loop over and hybridize to adjacent, complementary primers and are extended again to form two new double stranded loops. The process is repeated on each template copy by cycles of isothermal denaturation and amplification to create dense clonal clusters. Each cluster of double stranded bridges is denatured. In an example, the reverse strand is removed by specific cleavage at the cleavage site (e.g., uracil, 8oxoguanine, allyl-T, etc. in the primer sequence, leaving forward template strands. Clustering results in the formation of several template strands immobilized on the polymeric hydrogel through the primer. In one example, of the amplification that may be performed through a bridge amplification technique. It is to be understood that other amplification techniques may be used, including, e.g., exclusion amplification (ExAmp).

Some examples of the method then include blocking non-protected (free) 3′ OH ends of the template strands and primers that do not have template strands attached thereto. A blocking group (e.g., a 3′ phosphate) may be added that attaches to the exposed 3′ ends to prevent undesired extension.

Sequencing primers may then be introduced to the flow cell. The sequencing primers hybridize to the template nucleic acid strands. These sequencing primers render the template strandsready for sequencing.

An incorporation mix including labeled nucleotides may then be introduced into the flow cell, e.g., via the inlet. In addition to the labeled nucleotides, the incorporation mix may include water, a buffer, and polymerases. Referring to FIG. 1A, when the incorporation mix is introduced into the flow cell 10, the mix enters the flow channel 12, and contacts the anchored and sequence ready template strands.

The incorporation mix is allowed to incubate in the flow cell 10, and labeled nucleotides (including optical labels) are incorporated by respective polymerases into the nascent strands along the template strands. During incorporation, one of the labeled nucleotides is incorporated, by a respective polymerase, into one nascent strand that extends one sequencing primer and that is complementary to one of the template strands. Incorporation is performed in a template strand dependent fashion, and thus detection of the order and type of labeled nucleotides added to the nascent strand can be used to determine the sequence of the template strand. Incorporation occurs in at least some of the template strands across the flow cell 10 during a single sequencing cycle.

The incorporated labeled nucleotides may include a reversible termination property due to the presence of a 3′ OH blocking group, which terminates further sequencing primer extension once the labeled nucleotide has been added. After a desired time for incubation and incorporation, the incorporation mix, including nonincorporated labeled nucleotides, may be removed from the flow cell 10 during a wash cycle. The wash cycle may involve a flow-through technique, where a washing solution (e.g., buffer) is directed into, through, and then out of flow channel 12, e.g., by a pump or other suitable mechanism.

Without further incorporation taking place, the most recently incorporated labeled nucleotides can be detected through an imaging event. During the imaging event, an illumination system may provide an excitation light to the flow cell 10. The optical labels of the incorporated labeled nucleotides emit optical signals in response to the excitation light. These optical signals may be captured using an imaging device.

After imaging is performed, a cleavage mix may then be introduced into the flow cell 10. In an example, the cleavage mix is capable of i) removing the 3′ OH blocking group from the incorporated nucleotides, and ii) cleaving the optical label from the incorporated nucleotide. Examples of 3′ OH blocking groups and suitable deblocking agents/components in the cleavage mix may include: ester moieties that can be removed by base hydrolysis; allyl-moieties that can be removed with Nal, chlorotrimethylsilane and Na₂S₂O₃ or with Hg(II) in acetone/water; azidomethyl which can be cleaved with phosphines, such as tris(2-carboxyethyl)phosphine (TCEP) or tri(hydroxypropyl)phosphine (THP); acetals, such as tert-butoxy-ethoxy which can be cleaved with acidic conditions; MOM (—CH₂OCH₃) moieties that can be cleaved with LiBF₄ and CH₃CN/H₂O; 2,4-dinitrobenzene sulfenyl which can be cleaved with nucleophiles such as thiophenol and thiosulfate; tetrahydrofuranyl ether which can be cleaved with Ag(I) or Hg(II); and 3′ phosphate which can be cleaved by phosphatase enzymes (e.g., polynucleotide kinase). Examples of suitable optical label cleaving agents/components in the cleavage mix may include: sodium periodate, which can cleave a vicinal diol; phosphines, such as tris(2-carboxyethyl)phosphine (TCEP) or tri(hydroxypropyl)phosphine (THP), which can cleave azidomethyl linkages; palladium and THP, which can cleave an allyl; bases, which can cleave ester moieties; or any other suitable cleaving agent of the 3′ OH blocking group.

Additional sequencing cycles may then be performed until the template strands 40 are sequenced. The nascent strands may be dehybridized, and the blocking group at the 3′ OH ends of the template strands and primers may be removed. Clustering is performed again, and this time, the forward strands are removed by specific cleavage at the cleavage site (e.g., uracil, 8-oxoguanine, allyl-T, etc. in the primer sequence, leaving the reverse template strands. Sequencing of the reverse template strands may be performed as described herein.

After sequencing, the cleaving fluid is introduced into the flow cell 10, e.g., via the inlet, to cleave the grafted plurality of primers at the thioester-cysteine bond, thereby leaving a plurality of thioester functional groups at the surface of the flow cell 10. After a desired time for cleaving, a wash cycle may be performed to remove the cleaved portions.

With multiple reactive sulfur-containing functional groups again located at the surface of the polymeric hydrogel, the flow cell surface is ready for another round of primer grafting and nucleic acid analysis. The processes shown and described in reference to FIG. 2 may be repeated as desired to perform multiple nucleic acid analyses.

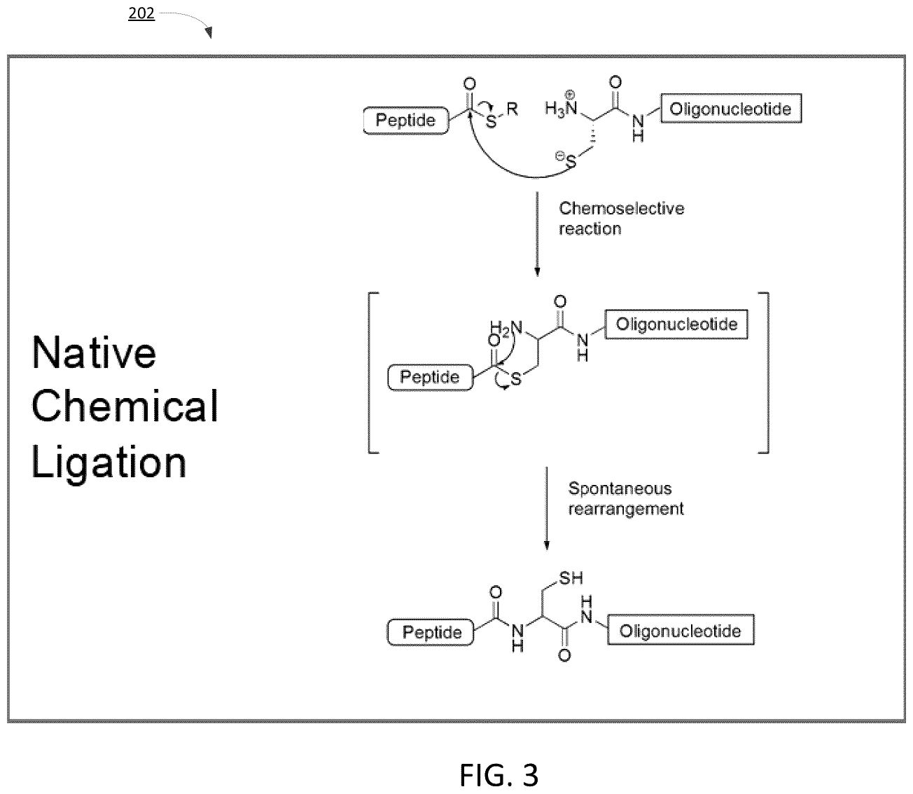

FIG. 3 shows an example two-step mechanism of the native chemical ligation step of FIG. 2 at 202, i.e., the reaction between a thioester and a terminal cysteine residue. As shown in FIG. 3, in native chemical ligation, an ionized thiol group of an N-terminal cysteine residue of an unprotected peptide may attack the C-terminal thioester of a second unprotected peptide. For example, native chemical ligation may occur in an aqueous buffer at pH 7.0 and room temperature. This transthioesterification may be reversible in the presence of an aryl thiol catalyst, rendering the reaction both chemoselective and regioselective, and may lead to formation of a thioester-linked intermediate. The intermediate may (e.g., rapidly and spontaneously) rearrange by an intramolecular S,N-acyl shift that results in the formation of a native amide (e.g., peptide) bond at the ligation site. The efficiency of this coupling reaction is typically near quantitative and the product of this reaction may be stable amide bond, which can be used for primer grafting.

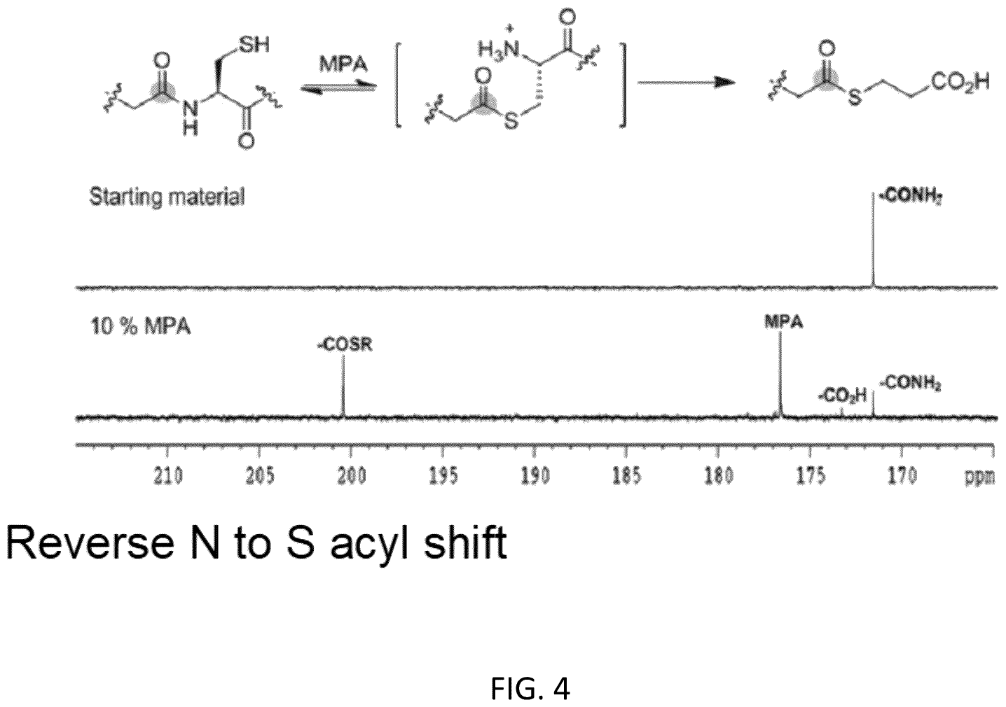

A native chemical ligation reaction may be reversible. For example, a native chemical ligation product may undergo an N-to-S acyl shift reaction when catalyzed by MPA, which may lead to fragmentation and may yield a C-terminus with a thioester. A thioester flow cell surface may be regenerated via the N-to-S acyl shift reaction after clustering and sequencing. Primers may then be grafted to the regenerated thioester flow cell surface, for example as described herein. FIG. 4 illustrates an example N-to-S acyl shift reaction. For example, an N-to-S acyl shift reaction may be performed using a solution containing 10% v/v acetic acid (AcOH), 10% w/v MESNa (sodium salt of 2-(N-morpholino)ethanesulfonic acid, and/or 0.5% w/v TCEP (tris(2-carboxyethyl)phosphine) at 60 degrees Celsius.

Various embodiments of methods in accordance with the present disclosure, may include the use of thiol and disulfide reactions. For example, various embodiments may include: providing a flow cell having a substrate, the substrate having a plurality of primer binding sites, each of the plurality of primer binding sites comprising a thiol or disulfide moiety; grafting a plurality of pyridyl-disulfide-terminated primers or thiol-terminated primers, respectively, to the primer binding sites thus forming a disulfide bond between the primer and the functionalized surface; performing a nucleic acid analysis; introducing a cleaving fluid to cleave the grafted plurality of primers at the disulfide bond, thereby leaving a plurality of thiol moieties at the surface of the flow cell; optionally contacting the surface of the flow cell with a regeneration fluid comprising a dipyridyl disulfide to provide multiple new pyridyl disulfide moieties on the surface of the flow cell; and optionally repeating these steps one or more times.

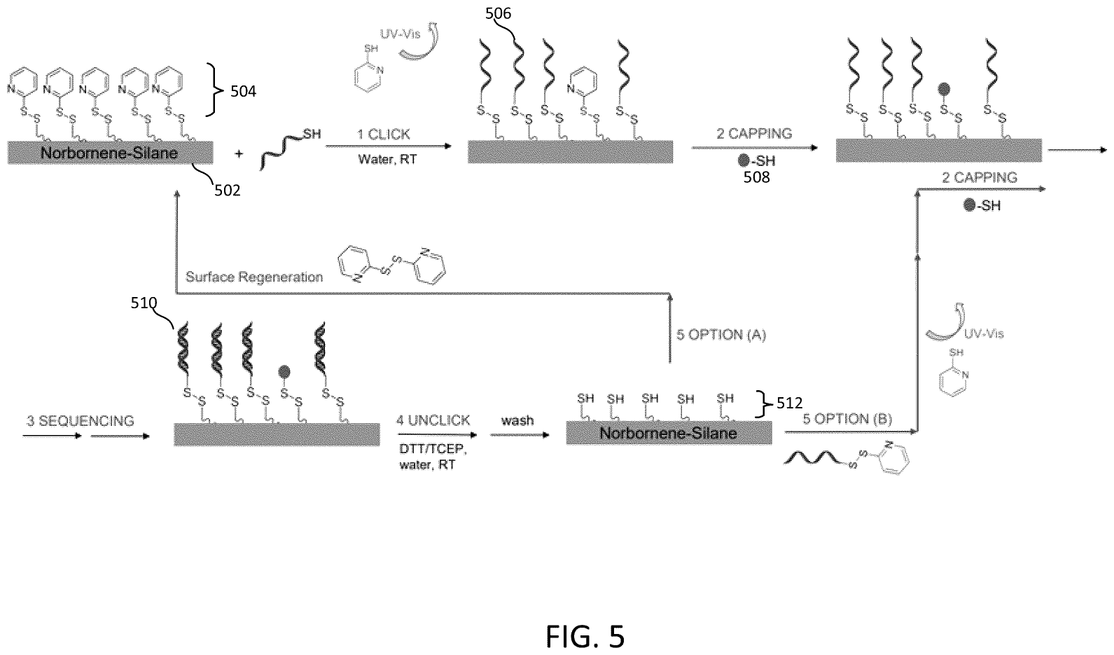

FIG. 5 illustrates examples of reversible oligonucleotide grafting for flow cell reuse via disulfide/thiol reactions. For example, as shown in FIG. 5, a flow cell 502 may be provided. The flow cell 502 may comprise a surface that is comprised of norbornene and/or silane. The flow cell 502 surface or polymer on the surface of the flow cell may further comprise one or more terminal pyridyl disulfide functional groups 504. One or more oligonucleotides comprising a terminal thiol functional group may be introduced to the flow cell surface, and a disulfide substitution reaction (e.g., a click reaction) may occur. The reaction may be performed with water as a solvent and at room temperature. The reaction results in the oligonucleotide primers 506 being grafted to the flow cell surface via a disulfide bond and in released 2-mercaptopyridine which can be monitored for quantitative purposes as discussed hereinbelow. The PDS reaction may be monitored, for example by measuring an ultraviolet (UV)/visible light signal generated by the released 2-mercaptopyridine which produces a measurable output at a wavelength of 365 nm.

As shown in FIG. 5, any remaining unreacted pyridyl disulfide groups may be capped. For example, the unreacted PDS groups may be capped with a thiol 508. A nucleotide analysis may then be carried out. After analysis (“sequencing” in FIG. 5) is performed, the sequenced DNA 510 may be removed via a second reaction that includes DTT (dithiothreitol) and/or TCEP (tris(2-carboxyethyl)phosphine), and water as a solvent at room temperature. The second reaction may result in the flow cell surface having one or more terminal thiol functional groups 512. As shown in FIG. 5, the flow cell surface may be contacted with a dipyridyldisulfide, such that the flow cell surface is again provided with pyridyl disulfide functional groups (e.g., option (A) shown in FIG. 5). Alternatively, one or more pyridyl disulfide-terminated oligonucleotide primers may be introduced to the flow cell surface, such that the flow cell surface has one or more oligonucleotides bonded to it (e.g., option (B) shown in FIG. 5). As shown in FIG. 5, the reaction carried out in Option B may also be quantitatively monitored using UV/Vis spectroscopy.

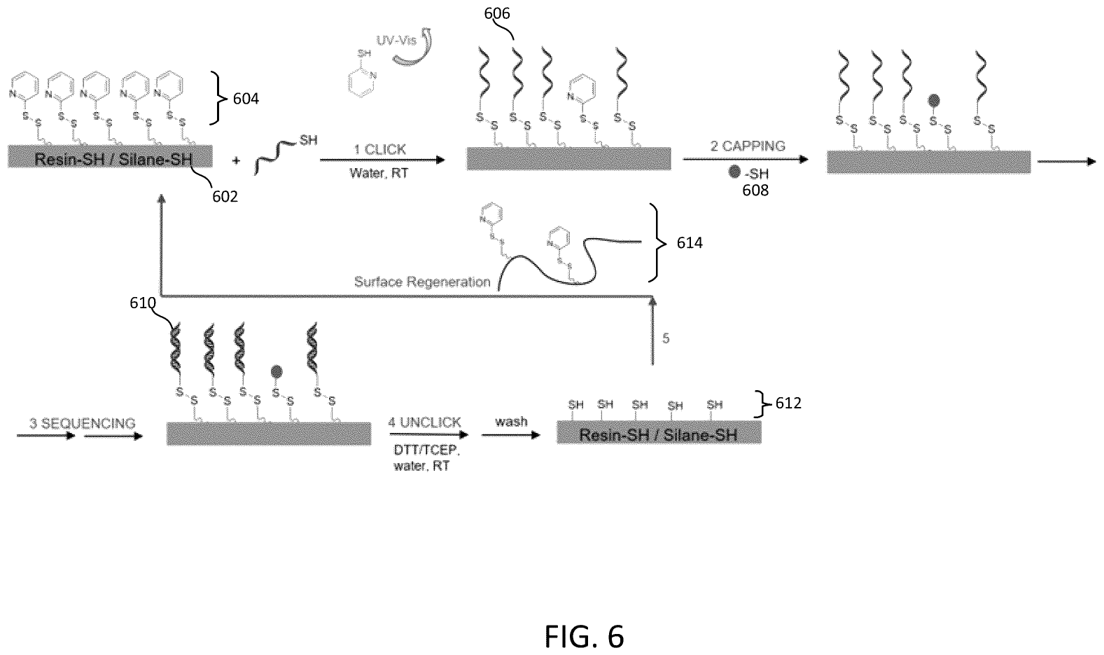

FIG. 6 illustrates an example of reversible polymer grafting for flow cell reuse via disulfide/thiol reactions. For example, as shown in FIG. 6, a flow cell 602 may be provided. The flow cell 602 may comprise a surface that is comprised of resin-sulfur hydride and/or silane-sulfur hydride, to which pyridyl disulfide 604 may be bound through the disulfide bridges. It is to be understood that the exemplary methods described in reference to FIG. 5 may be performed using polymers bearing pendant pyridyl disulfide groups as described herein with reference to FIG. 6, and conversely, the polymers bearing pendant pyridyl disulfide groups described herein with reference to FIG. 6 can be used, for example, in Option A described above. One or more thiol-terminated oligonucleotide primers may be introduced to the flow cell surface, and a thiol/disulfide reaction (e.g., a click reaction) may occur. The reaction may be performed with water as a solvent and at room temperature. The reaction results in oligonucleotide primers 606 being grafted to the flow cell surface via a disulfide bond and in released 2-mercaptopyridine. The PDS reaction may be monitored, for example by measuring an ultraviolet (UV)/visible light signal generated by the released 2-mercaptopyridine.

As shown in FIG. 6, any remaining unreacted pyridyl disulfide groups may be capped with a thiol 608 as discussed above. A nucleic acid analysis/DNA sequencing may be performed on the attached oligonucleotides. After sequencing is performed, the sequenced DNA 610 may be removed via a second reaction that includes DTT/TCEP and water as a solvent at room temperature. The second reaction may result in the flow cell surface having one or more thiol functional groups 612. As shown in FIG. 6, the flow cell surface may be contacted with a polymer 614 having pendant pyridyl disulfide groups, such that the flow cell surface is provided with pyridyl disulfide functional groups. For example, the polymer may have pendant pyridyl disulfide groups for reaction with the thiol moieties and to provide the resulting surface with reactive pyridyl disulfide moieties.

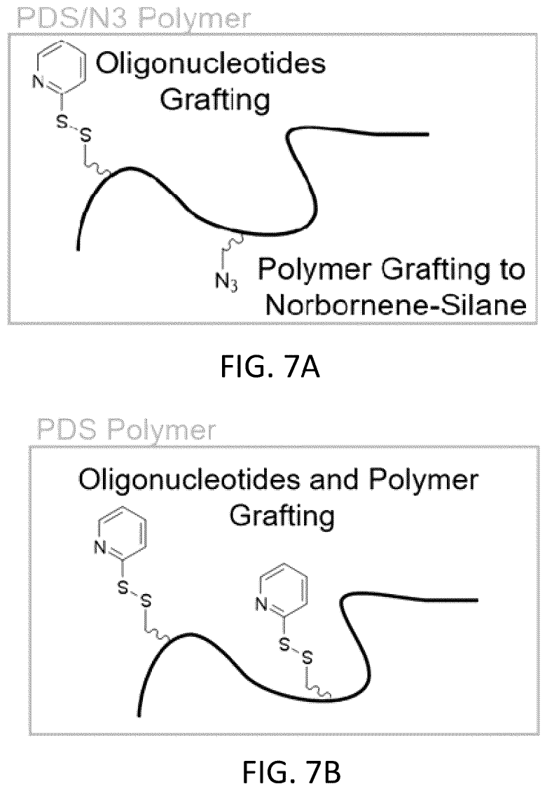

Pyridyl disulfide reactions (PDS) employing polymers with pendant pyridyl disulfide groups may be used for reversible oligonucleotide grafting and reversible polymer grafting on a variety of surfaces. For example, FIG. 7A illustrates an example of a PDS polymer that can be employed on a norbornene/silane flow cell surface. The polymer has pendant azide groups in addition to pendant pyridyl disulfide groups. Azide/norbornene click chemistry may be used for polymer grafting. Thiol-terminated primers may then be bound to the surface by reversible reaction with the disulfide. FIG. 7B shows an example of a PDS polymer that can be employed on a thiol-containing resin and/or thiol-functionalized surface. The pendant pyridyl disulfide groups can react with the surface thiols and provide primer binding sites as the reaction is controllable and may be monitored with UV/Vis spectroscopy via the release of 2-mercaptopyridine.

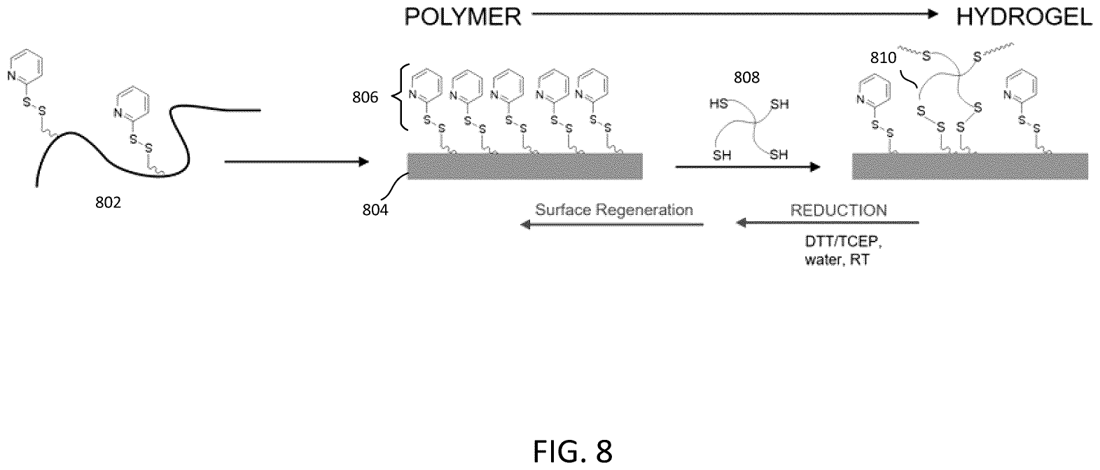

FIG. 8 illustrates an example of a cross-linking reaction (e.g., a reversible cross-linking reaction) using PDS. PDS-containing polymers may be used to generate a fast and reversible in situ cross-linking by adding molecules containing multiple thiol functionalities. For example, 2/4-arm-thiol-containing polymers may be used. As shown in FIG. 8, a PDS-containing molecule 802 may be introduced to the surface of a flow cell 804 such that the flow cell 804 surface has one or more terminal PDS functional groups 806. For example, the PDS-containing molecule may be a polymer having two or more terminal PDS functional groups. A molecule 808 containing multiple thiol functionalities may be introduced to the flow cell surface, such that the flow cell surface has one or more cross-linked thiol groups 810. As shown in FIG. 8, the cross-linked thiol groups may be removed from the flow cell surface via a reduction reaction. For example, the reduction reaction may be performed using DTT/TCEP in water, at room temperature. The flow cell 804 surface may then be regenerated by adding a PDS-containing molecule to the flow cell 804 surface.

Reference throughout the specification to “one example”, “another example”, “an example”, and so forth, means that a particular element (e.g., feature, structure, and/or characteristic) described in connection with the example is included in at least one example described herein, and may or may not be present in other examples. In addition, it is to be understood that the described elements for any example may be combined in any suitable manner in the various examples unless the context clearly dictates otherwise.

It will be appreciated by those skilled in the art that changes could be made to the embodiments described above without departing from the broad concepts thereby illustrated. It is understood, therefore, that the disclosure is not limited to the particular embodiments illustrated herein, but it is intended to cover modifications within the spirit and scope of the present disclosure as defined by the appended claims.

What is claimed is:

1. A flow cell comprising a substrate, the substrate comprising a plurality of primer binding sites, each of the plurality of primer binding sites comprising a reactive sulfur moiety.

2. The flow cell according to claim 1, wherein the reactive sulfur moiety comprises a moiety selected from the group consisting of thiols, thioesters, and disulfides.

3. The flow cell according to claim 1, wherein the reactive sulfur moiety comprises a thiol.

4. The flow cell according to claim 1, wherein the reactive sulfur moiety comprises a thioester.

5. The flow cell according to claim 1, wherein the reactive sulfur moiety comprises a disulfide.

6. The flow cell according to claim 1, wherein the reactive sulfur moiety comprises a pyridyl disulfide.

7. A method comprising:
providing a flow cell having a substrate comprising a plurality of primer binding sites, each of
the plurality of primer binding sites comprising a first reactive sulfur moiety;
grafting oligonucleotide primers to a portion of the plurality of primer binding sites;
performing a nucleic acid sequence analysis; and
contacting the flow cell substrate with a cleaving reagent that removes the oligonucleotide primers such that the substrate comprises a plurality of primer binding sites comprising a second reactive sulfur moiety.

8. The method according to claim 7, wherein the first reactive sulfur moiety and the second reactive sulfur moiety are the same moiety.

9. The method according to claim 8, wherein the first reactive sulfur moiety and the second reactive sulfur moiety comprise thioesters, and wherein the oligonucleotide primers comprise cysteine terminated primers.

10. The method according to claim 8, wherein the first reactive sulfur moiety and the second reactive sulfur moiety comprise thiols, and wherein the oligonucleotide primers comprise pyridyl disulfide terminated primers.

11. The method according to claim 7, wherein the first reactive sulfur moiety comprises a disulfide and the second reactive sulfur moiety comprises a thiol, and wherein the method further comprises reacting the thiols with dipyridyl disulfide to reform a plurality of primer binding sites comprising disulfides.

12. The method according to claim 7, wherein the first reactive sulfur moiety comprises a disulfide and the second reactive sulfur moiety comprises a thiol, and wherein the method further comprises reacting the thiols with a polymer having pendant pyridyl disulfide moieties to reform a plurality of primer binding sites comprising disulfides.

13. The method according to claim 7, wherein the first reactive sulfur moiety comprises a disulfide and the second reactive sulfur moiety comprises a thiol, wherein the oligonucleotide primers comprise pyridyl disulfide terminated primers, and wherein the method further comprises a subsequent sequencing cycle wherein the thiols are contacted with pyridyl disulfide terminated primers and a subsequent nucleic acid sequence analysis is carried out.

14. The method according to claim 7, wherein the grafting of oligonucleotide primers or contacting of the flow cell with the cleaving agent is monitored by measuring ultraviolet-visible (UV-Vis) signal generation.

15. (canceled)

16. (canceled)

17. (canceled)

18. (canceled)

19. (canceled)

20. A kit comprising:
a flow cell having a substrate comprising a plurality of primer binding sites, each of the plurality of primer binding sites comprising a first reactive sulfur moiety;
a priming reagent comprising oligonucleotide primers having a terminal group that forms a bond with the primer binding sites; and
a cleaving reagent that is capable of removing the oligonucleotide primers such that the substrate comprises a plurality of primer binding sites comprising a second reactive sulfur moiety.
